SEQUENCE LISTING (23 sequences)

SEQ ID NO: 1 - protein, 9 aa, synthetic construct
CAEECRPIA

SEQ ID NO: 2 - protein, 15 aa, synthetic construct
GEPPPGKPADDAGLV

SEQ ID NO: 3 - protein, 9 aa, synthetic construct
SLLGRMKGA

SEQ ID NO: 4 - protein, 29 aa, synthetic construct
YADAIFTNSYRKVLGQLSARKLLQDIMSR

SEQ ID NO: 5 - protein, 9 aa, synthetic construct
WAGGDASGE

SEQ ID NO: 6 - protein, 4 aa, synthetic construct
RKLH

SEQ ID NO: 7 - protein, 4 aa, synthetic construct
AWFK

SEQ ID NO: 8 - protein, 5 aa, synthetic construct
WAWFK

SEQ ID NO: 9 - protein, 29 aa, synthetic construct
YADAIFTNSYRKVLGQLSARKLLQDIMSR

SEQ ID NO: 10 - protein, 24 aa, synthetic construct
MAPRGFSCLLLLTSEIDLPVKRRA

SEQ ID NO: 11 - protein, 10 aa, synthetic construct
YNWNSFGLRF

SEQ ID NO: 12 - protein, 37 aa, synthetic construct
LLGDFFRKSKEKIGKEFKRIVQRIKDFLRNLVPRTES

SEQ ID NO: 13 - protein, 16 aa, synthetic construct
MRWQEMGYIFYPRKLR

SEQ ID NO: 14 - protein, 4 aa, synthetic construct
AEDG

SEQ ID NO: 15 - protein, 8 aa, synthetic construct
TPLVTLFK

SEQ ID NO: 16 - protein, 7 aa, synthetic construct
VRSSSRT

SEQ ID NO: 17 - protein, 32 aa, synthetic construct
PPLSQETFSDLWKLLKKWKMRRNQFWVKVQRG

SEQ ID NO: 18 - protein, 27 aa, synthetic construct
ETFSDLWKLLKKWKMRRNQFWVKVQRG

SEQ ID NO: 19 - protein, 20 aa, synthetic construct
KRRQTSMTDFYHSKRRLIFS

SEQ ID NO: 20 - protein, 28 aa, synthetic construct
SDAAVDTSSEITTKDLKEKKEVVEEAEN

SEQ ID NO: 21 - protein, 28 aa, synthetic construct
HSDAVFTDNYTRLRKQMAVKKYLNSILN

SEQ ID NO: 22 - protein, 43 aa, synthetic construct
SDKPDMAEIEKFDKSKLKKTETQEKNPLPSKETIEQEKQAGES

SEQ ID NO: 23 - protein, 9 aa, synthetic construct
KTKKEAAKK

(19) United States
(12) Patent Application Publication
(10) Pub. No.: US 2025/0360156 A1
(43) Pub. Date: Nov. 27, 2025

(54) SYNERGISTIC COMBINATION OF NAD+ AND THERAPEUTIC PEPTIDES DELIVERED VIA IONTOPHORESIS FOR ENHANCED AND PROLONGED REGENERATIVE EFFECTS

(71) Applicants: Nicholas Jerome Andrews, Flanders, NJ (US); Roy Charles Korth, Grove City, OH (US); Charles David Sly, Grove City (OH)

(72) Inventors: Nicholas Jerome Andrews, Flanders, NJ (US); Roy Charles Korth, Grove City, OH (US); Charles David Sly, Grove City (OH)

(21) Appl. No.: 19/186,065

(22) Filed: Apr. 22, 2025

Related U.S. Application Data
(60) Provisional application No. 63/636,901, filed on Apr. 22, 2024.

(51) Int. Cl. A61K 31/7076; A61K 9/00; A61K 38/06; A61K 38/17; A61K 47/12; A61N 1/04

(52) U.S. Cl. CPC A61K 31/7076; A61K 9/0009; A61K 38/06; A61K 38/1774; A61K 47/12; A61N 1/0432

(57) ABSTRACT

The present disclosure also provides for a composition comprising a combination of (a) NAD+, and (b) one or more additional therapeutic compounds, wherein one or more additional therapeutic compounds are compatible with transdermal delivery, such as iontophoretic delivery, while providing minimal skin irritation.

### CROSS-REFERENCE TO RELATED APPLICATIONS

The present disclosure claims the benefit of the filing dates of U.S. Provisional Application No. 63/614, 144 filed on Dec. 22, 2023

### FIELD OF DISCLOSURE

The present disclosure relates to the field of regenerative medicine and drug delivery systems. Specifically, the present disclosure pertains to the delivery of nicotinamide adenine dinucleotide (NAD+) in combination with one or more therapeutic peptides, peptide bioregulators, or compounds that are believed to facilitate cellular respiration and or regenerative cellular pathways and regenerative signaling.

### SEQUENCE LISTING

The contents of the electronic sequence listing (PushPatch.xml sequence listing.xml; Size: 20,808 bytes; and Date of Creation: Apr. 21, 2025) is herein incorporated by reference in its entirety.

### BACKGROUND

Regenerative medicine has made significant strides in recent years, with the discovery of various peptides, peptide bioregulators, and compounds that promote tissue repair, wound healing, and cellular regeneration. These agents exert their effects by modulating inflammatory responses, stimulating angiogenesis, promoting cell proliferation and migration, and enhancing cellular respiration. However, the efficacy of these regenerative agents is often limited by their short half-lives, ranging from minutes to a few hours, which necessitates frequent administration and limits their therapeutic potential.

### SUMMARY OF THE DISCLOSURE

The present disclosure addresses the limitations of existing regenerative therapies by providing a composition comprising a combination of (a) NAD+, and (b) one or more (i) therapeutic peptides, (ii) peptide bioregulators, and/or (iii) compounds that are believed to facilitate cellular respiration and or regenerative cellular pathways (e.g., cellular respiration enhancers) and cellular regenerative pathways.

Clinical and mechanistic studies demonstrate that co-delivery of NAD⁺ with regenerative peptides, such as BPC-157, yields synergistic therapeutic outcomes in tissue injuries marked by mitochondrial dysfunction, energy depletion, and impaired repair signaling—particularly in ischemic contexts like myocardial infarction or chronic non-healing wounds (Wang et al., 2021; Covarrubias et al., 2021).

In ischemic events, rapid NAD⁺ depletion compromises mitochondrial ATP generation, PARP-mediated DNA repair, and the activity of NAD⁺-dependent enzymes (e.g., sirtuins, CD38), precipitating cell death, diminished angiogenesis, and persistent inflammation. Concurrently, BPC-157—a stable gastric pentadecapeptide—potentiates angiogenesis and tissue remodeling via upregulation of VEGF and eNOS signaling, while mitigating oxidative stress through nitric oxide modulation and pro-resolving cytokine cascades.

By providing sustained transdermal NAD⁺ via anodal iontophoresis in tandem with BPC-157, the present disclosure uniquely addresses both energetic deficits and reparative blockade. NAD⁺ replenishment restores intracellular ATP, supports sirtuin-driven cytoprotective gene expression, and maintains mitochondrial redox balance—thereby enabling effective utilization of BPC-157's reparative signaling, which is metabolically demanding and reliant on intact NAD⁺ pools for transcriptional and translational fidelity (Poljšak et al., 2023).

Preclinical models corroborate this dual mechanism. In muscular dystrophy subjects, NAD⁺ repletion improved mitochondrial integrity and stem cell regenerative capacity—effects mechanistically linked to reduced global PARylation and restored SIRT1/SIRT3 activity (Zhang et al., 2016). Likewise, BPC-157 has been shown to stabilize endothelial function and attenuate ischemia-reperfusion injury, although its efficacy is markedly diminished under NAD-depleted conditions where cellular energy stores are compromised.

The present disclosure is directed to the sustained, co-localized iontophoretic delivery of NAD⁺ and peptide therapeutics over a predetermined time period, such as a time period ranging from about 10 hours to about 16 hours, such as about 12 hours to about 4 hours, thereby bypassing hepatic first-pass metabolism and circumventing the inefficiencies of precursor-only approaches (e.g., NMN, NR) that rely on intracellular enzymatic conversion and which may not rapidly restore mitochondrial NAD⁺ under acute injury. This co-delivery modality ensures continuous energetic support for peptide-mediated signaling, enabling enhanced infarct-zone angiogenesis, accelerated wound granulation, and improved functional recovery metrics—potentially reducing infarct size or non-healing wound burden by between about 25—to about 35% relative to monotherapy regimens.

The present disclosure also provides for a composition comprising a combination of (a) NAD+, and (b) one or more additional therapeutic compounds, wherein one or more additional therapeutic compounds are compatible with transdermal delivery, such as iontophoretic delivery, while providing minimal skin irritation. Iontophoresis is defined as the use of electric current to drive molecules across cell membranes through an electrolyte solution. In therapeutic context, it is used to facilitate the administration of bioactive substances, either systemically or locally. In some embodiments, the one or more additional therapeutic compounds are therapeutic peptides, peptide bioregulators, and/or compounds that are believed to facilitate cellular respiration and or regenerative cellular pathways. In some embodiments, the one or more additional therapeutic compounds may include, but are not limited to, charged small molecules, peptides, or biologics that benefit from improved mitochondrial function or energy metabolism, such as anti-inflammatory agents, analgesics, antioxidants, or growth factors, provided they possess a net charge under optimized pH conditions suitable for iontophoretic transport. In some embodiments, the compositions have a pH ranging from about 4.8 to about 5.5 to facilitate anodic delivery through an iontophoretic device. In other embodiments, the compositions have a pH ranging from between about 7.5 to about 8.0 to facilitate cathodic delivery through an iontophoretic device. In some embodiments, the inclusion of such one or more additional therapeutic compounds leverages the synergistic enhancement of cellular respiration provided by NAD+ and related components, enabling a broader range of regenerative or therapeutic outcomes when delivered transdermally via an iontophoretic system.

Another aspect of the present disclosure is an iontophoretic delivery system including a composition comprising a combination of (a) NAD+, and (b) one or more (i) therapeutic peptides, (ii) peptide bioregulators, and/or (iii) compounds that are believed to facilitate cellular respiration and or regenerative cellular pathways. Wishing to be bound by any particular, it is believed that this approach aims to enhance and prolong the regenerative effects of these agents by providing a sustained delivery system, optimizing cellular respiration, and promoting tissue repair and regeneration.

Another aspect of the present disclosure is a system for iontophoretic transdermal delivery including an iontophoresis device comprising at least one agent reservoir adapted for holding a composition including a combination of (a) NAD+, and (b) one or more (i) therapeutic peptides, (ii) peptide bioregulators, and/or (iii) compounds that are believed to facilitate cellular respiration and or regenerative cellular pathways (e.g., cellular respiration enhancers). In some embodiments, the compositions have a pH ranging from about 4.8 to about 5.5 to facilitate anodic delivery through an iontophoretic device. In other embodiments, the compositions have a pH ranging from between about 7.5 to about 8.0 to facilitate cathodic delivery through an iontophoretic device. Suitable iontophoresis devices and components of such iontophoresis devices include those described in U.S. Publication Nos. 20050070840, 20070066932, and 20090221985; and U.S. Pat. Nos. 7,945,320 and 9,492,650 the disclosures of which are hereby incorporated by reference herein in their entireties.

The present disclosure also provides a kit comprising (i) a composition comprising a combination of (a) NAD⁺, and (b) one or more (i) therapeutic peptides, (ii) peptide bioregulators, and/or (iii) compounds that facilitate cellular respiration and or regenerative cellular pathways; and (ii) an iontophoretic delivery device. In some embodiments, the iontophoretic delivery device comprises a reservoir for storing the composition, at least one electrode, and an electrical energy source. In other embodiments, the device includes two electrodes and an integrated power module, all in a self-contained, wearable format. Suitable iontophoresis devices and components of such iontophoresis devices include those described in U.S. Publication Nos. 20050070840, 20070066932, and 20090221985; and U.S. Pat. Nos. 7,945,320 and 9,492,650 the disclosures of which are hereby incorporated by reference herein in their entireties.

Another aspect of the present disclosure is a fully integrated, single-use iontophoresis patch system employed for the transdermal co-delivery of NAD⁺ and one or more (i) therapeutic peptides, (ii) peptide bioregulators, and/or (iii) compounds that facilitate cellular respiration and or regenerative cellular pathways (such as peptides such as BPC-157 or KPV). In some embodiments, the patch incorporates both an anode and a cathode, each pre-coated with the appropriate electrode material, and houses a built-in galvanic power source that activates upon hydration. In some embodiments, delivery proceeds via low-intensity direct current (DC) over approximately 14 hours, as exemplified by systems described in U.S. Pat. Nos. 6,653,014 B2 and 6,745,071 B1. Such patches, it is believed, facilitate consistent current density (e.g., 0.05-0.1 mA/cm²), obviating external wiring and/or batteries.

In some embodiments, the iontophoresis patch system is the IontoPatch® platform. The IontoPatch® platform comprises:
Electrodes: a zinc anode and a silver/silver-chloride cathode for stable ion generation (U.S. Pat. Nos. 6,421,561; 6,653,014; 6,745,071; 7,016,723; 7,031,768; 7,031,769).
Drug Reservoir: a hydrated hydrogel matrix pre-loaded with the therapeutic formulation, positioned in contact with the appropriate electrode according to drug polarity.
Power Source: an embedded galvanic cell that initiates upon skin contact, generating a controlled DC current for the patch's lifetime.—Adhesive and Backing Layers: biocompatible adhesive for secure placement and a flexible, moisture-resistant backing to protect internal components.

In some embodiments, the iontophoresis patch system is the ActivaPatch® system (ActivaTek Inc.). The ActivaPatch® system offers both single-use and reusable configurations:
Electrodes: zinc anode and silver/silver-chloride cathode optimized for ionic conductivity (U.S. Pat. Nos. 6,775,570; 7,047,069; 8,197,844).
Drug Reservoir: disposable pre-filled gel cartridges (single-use) or replaceable drug modules (reusable) containing ionic therapeutics.
Power Source: in single-use models, a self-contained galvanic cell; in reusable models, a detachable battery-powered controller delivering adjustable DC current (up to 4 mA) with safety shut-off on dose completion.
Adhesive and Backing: medical-grade adhesive interfaces and robust, water-resistant backing for both disposability and multiple-use reliability

While IontoPatch® and ActivaPatch® illustrate embodiments of single-use and reusable iontophoretic devices, the scope of the disclosure encompasses any iontophoresis platform—whether fully integrated patches, modular systems, or hybrid designs—that employs comparable electrode configurations, hydration-activated power sources, and controlled DC delivery profiles for the co-delivery of NAD⁺ or its precursors in combination with one or more therapeutic peptides, peptide bioregulators, or compounds that are believed to facilitate cellular respiration and or regenerative cellular pathways and regenerative signaling.

The present disclosure also provides a kit comprising (i) a composition comprising a combination of (a) NAD+, and (b) one or more (i) therapeutic peptides, (ii) peptide bioregulators, and/or (iii) compounds that are believed to facilitate cellular respiration and or regenerative cellular pathways; and (ii) an iontophoretic delivery circuit. In some embodiments, the iontophoretic delivery circuit comprises at least two electrodes and an electrical energy source (e.g., a battery).

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of,” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

### Overview

A primary difficulty in employing iontophoresis for delivering a composition including a combination of NAD+ and one or more additional therapeutic compounds (e.g., one or more (i) therapeutic peptides, (ii) peptide bioregulators, and/or (iii) compounds that are believed to facilitate cellular respiration and or regenerative cellular pathways or initiate regenerative cellular signaling) lies in the pH-dependent charge behavior of the constituent molecules, which directly affects their transport efficiency.

Iontophoresis involves the use of low-intensity current (usually <0.5 mA/cm²) to transport both charged and neutral species into the skin, based on two main mechanisms of action: electromigration and electro-osmosis. Electromigration is based on the principle that like charges repel each other. As such, iontophoresis relies on the principle of like charges repelling—positively charged molecules are driven from the anode (positive electrode), while negatively charged molecules are driven from the cathode (negative electrode).

During electromigration, charged molecules move under the influence of an electrical field, when in contact with an electrode of the same charge. Hence, when the electrical current is applied, cations are repelled by the anode and anions by the cathode, moving into the skin. In electro-osmosis, a bulk flow of fluid, also called solvent flow, is driven by a difference in electrical potential across a charged, porous membrane. The electrical field causes the free counter-ions of the charged membrane to migrate towards the oppositely charged electrode, carrying water molecules in the process. This results in a solvent flow that, in turn, carries neutral as well as charged molecules along with it, in the same direction. The direction of the flow depends on the charge in the biological membrane.

By way of example, NAD+ and its precursors, such as nicotinamide riboside (NR), exhibit variable charge states depending on pH. NR is neutral at a physiological pH of 7; but becomes positively charged at a pH of 5 due to protonation of its pyridine ring. Therapeutic peptides, such as BPC-157 or GHK-Cu, similarly possess pH-dependent charges influenced by their amino acid compositions, with net charges shifting from positive to neutral or negative as pH increases across their isoelectric points. Combining these “active” agents (i.e., NAD+ and BPC-157 or GHK-Cu) in a single formulation requires a pH that simultaneously maintains the appropriate charge state for each “active” component of the composition to enable effective iontophoretic transport from the same electrode, a task complicated by their differing chemical properties and optimal pH ranges.

This pH optimization is further constrained by the need to minimize skin irritation, a known limitation of iontophoretic systems. Excessively acidic (e.g., pH 5) or alkaline conditions can cause substantial skin irritation or burns, rendering the delivery system unacceptable to users. For instance, delivering NR at pH 5 to achieve a positive charge might enhance its transport from the anode but could irritate the skin, while a neutral pH of 7, safer for skin contact, renders NR uncharged and thus incompatible with iontophoresis. For instance, peptides like BPC-157, which may most effectively undergo, could conflict with NAD+'s requirements.

When formulating iontophoretic systems that co-deliver both NAD⁺ and BPC-157, the complexity of optimizing electrokinetic transport is significantly magnified due to the divergent charge architectures and transport mechanisms of the two compounds. NAD⁺, a highly anionic molecule under physiological conditions, typically carries a net charge between about 2 and about 3 at pH values compatible with skin tolerability (pH 5.0-7.0), driven primarily by its pyrophosphate moieties. In contrast, BPC-157 exhibits a distributed charge landscape, composed of both cationic (N-terminal and lysine) and anionic (glutamic acid, aspartic acid, and C-terminal) residues, resulting in a net charge of approximately 0.5 at pH 5.0. The differing electrochemical behavior of these two molecules necessitates precise control over pH, buffer composition, and ionic strength to ensure simultaneous and directional transport from a shared electrode interface.

This dual-agent configuration introduces a formulation constraint space that must harmonize the competing requirements for electrophoretic migration and electroosmotic facilitation. While NAD⁺, being strongly anionic, relies primarily on cathodal delivery or passive electroosmotic drag from the anode, BPC-157's partial compatibility with anodal delivery is mediated by its cationic subunits and net near-neutral charge at mildly acidic pH. Accordingly, a shared anodal delivery route is theoretically feasible but contingent on achieving a formulation pH that sufficiently protonates the phosphate groups of NAD⁺ to reduce net negative charge, while maintaining BPC-157 in a partially protonated state that preserves the positive charge on the lysine and N-terminal amine.

Moreover, the differential ion mobility of NAD⁺ and BPC-157 across the stratum corneum further complicates formulation design. The smaller molecular weight and higher charge density of NAD⁺ favor rapid migration under an electric field but may lead to competitive inhibition of BPC-157 transport due to ionic crowding and current partitioning. Conversely, excess buffering to suppress local pH shifts and reduce skin irritation—while beneficial from a tolerability standpoint—may attenuate the electrophoretic potential gradient necessary to drive either molecule effectively. These factors demand a carefully tuned formulation matrix that not only balances the subunit and net charge states of each therapeutic compound but also integrates skin-compatible pH and ionic environment optimization.

Therefore, co-formulation of NAD⁺ and BPC-157 for iontophoretic delivery represents a nontrivial electrochemical engineering problem wherein pH-dependent charge states, subunit-specific ionic interactions, and the interplay between electrophoresis and electroosmosis must be harmonized. The formulation must be sufficiently acidic to support partial protonation of NAD⁺ phosphate groups and preserve the cationic domains of BPC-157, yet buffered to remain within dermatologically acceptable tolerability thresholds. Such optimization strategies necessitate empirical titration of buffer systems, electrode polarity configuration, and total ionic strength to enable concurrent delivery with maximal bioavailability and minimal dermal irritation.

In the context of iontophoretic delivery, peptides such as BPC-157 exhibit complex charge behavior that must be accounted for in both formulation design and electrode selection. Although the molecule as a whole possesses a net charge—approximately −0.5 at pH 5.0—this aggregate value does not fully describe the electrokinetic behavior of the molecule under an applied current. Rather, BPC-157 comprises individual amino acid subunits, each of which maintains distinct acid-base equilibria and contributes independent ionic character to the overall molecule. Specifically, the side chains of glutamic acid (Glu) and aspartic acid (Asp), along with the C-terminal carboxyl group, retain negative partial charges at physiologically relevant pH values, including pH 5.0. These localized anionic centers contribute to the molecule's interaction with the electric field and may facilitate partial attraction to the anode through electroosmotic flow and localized electrophoretic interaction.

Conversely, the N-terminal amine and the s-amino group of the lysine (Lys) side chain are both positively charged at pH 5.0, each contributing a discrete +1 charge. These cationic regions establish localized domains of positive electrostatic potential that enhance compatibility with anodal migration. As a result, BPC-157's electrotransport behavior under iontophoresis is not solely dictated by its net molecular charge, but rather by a distributed charge architecture wherein distinct subunits exhibit opposing ionic tendencies. This charge heterogeneity enables complex interactions with both the applied electric field and the surrounding electrochemical environment, allowing partial alignment with anodal delivery despite a marginally negative overall charge.

The capacity for BPC-157 to undergo effective anodal transport is therefore driven by a dynamic interplay between electrophoretic mobility, electroosmotic drag, peptide conformation, and hydration shell dynamics. Electroosmosis—the solvent flux toward the cathode induced by the net cationic character of the skin—may co-transport neutrally or weakly negatively charged peptides such as BPC-157, especially when positively charged domains are present. Accordingly, the formulation must be optimized to exploit both charge distribution and solvent flow, allowing for enhanced transdermal transport from the anode despite an unfavorable net charge

This nuanced electrochemical profile underscores the necessity of formulating iontophoretic preparations in consideration of both whole-molecule net charge and the protonation states of individual amino acid residues. The Glu, Asp, and C-terminal moieties contribute negatively charged regions, while the N-terminal amine and lysine side chain contribute positively charged centers. The relative balance among these groups is highly pH-sensitive and determines the effective electrokinetic response of the molecule. Accordingly, buffer systems such as sodium citrate are employed to stabilize formulation pH and reduce skin irritation, but must be carefully balanced, as they alter ionic strength and may compete with the active agent for ionic mobility, thereby influencing both efficacy and tolerability of the delivery system

Identifying the proper electrode terminal (anode or cathode) for delivery introduces additional hurdles when combining NAD⁺ with one or more therapeutic compounds. In conventional single-agent systems—such as the patient-controlled fentanyl patch described in U.S. Pat. No. 5,697,896—which employs a single drug reservoir at one electrode and inert electrolyte at the other, only one ionic species is delivered, precluding concurrent administration of a second agent with opposing charge requirements. Similarly, U.S. Pat. No. 5,843,015 teaches modification of a single peptide to optimize its own iontophoretic transport but does not contemplate simultaneous transport of an additional therapeutic under a different polarity. Likewise, U.S. Pat. No. 4,383,529 utilizes a drug-loaded gel in the donor electrode and a plain electrolyte gel in the return electrode, with no provision for dual active reservoirs or charge-balancing strategies. Consequently, attempting to co-deliver NAD⁺ and a cationic peptide (e.g., GHK-Cu) alongside a neutral or anionic cofactor such as coenzyme Q10 would fall outside the scope of these single-agent designs and would necessitate complex dual-electrode or sequential delivery schemes incompatible with standard patch simplicity.

The interdependence of pH, charge, electrode selection, and stability create a multidimensional optimization problem that would not be readily apparent to the person of ordinary skill in the art. For instance, merely adjusting pH to favor NAD+ iontophoretic delivery might neutralize a peptide's charge, halting its transport; while buffering to protect skin might dilute the electric field's effectiveness, reducing overall delivery rates. The present disclosure overcomes the aforementioned formulation challenges through the development of a novel buffered iontophoretic composition specifically engineered to stabilize pH, minimize electrochemical irritation, and maintain directional transport efficacy for both NAD⁺ and BPC-157. Unlike prior art systems that treat single-agent delivery in isolation or disregard the pH-sensitivity of therapeutic peptides and cofactors, the disclosed formulation simultaneously accommodates the molecular charge complexities of both compounds across relevant physiological pH ranges. This is achieved by incorporating a carefully titrated buffering system—specifically, sodium citrate—into the formulation matrix, thereby mitigating the adverse electrochemical effects associated with prolonged iontophoretic application.

Although both NAD⁺ and BPC-157 contain structural subunits that contribute negatively charged domains under mildly acidic conditions (pH 5.0-6.0)—primarily from the phosphate groups of NAD⁺ and the Glu, Asp, and C-terminal residues of BPC-157—anodal delivery was selected and shown to be effective. This counterintuitive polarity configuration leverages the physiologic principle of electroosmotic flow, a bulk solvent movement from the anode toward the cathode, which can co-transport neutral or weakly anionic molecules along with the solvent front. In the context of human skin, which exhibits net negative fixed charges in the stratum corneum, electroosmosis predominates over electrophoresis in many practical cases of transdermal delivery. As a result, despite the partial anionic nature of both therapeutic agents, effective anodal transport was achieved through the synergistic action of electroosmotic drag and partial electrophoretic compatibility, particularly in the case of BPC-157, whose lysine and N-terminal amine residues retain discrete +1 charges at the target pH.

Mechanistically, sodium citrate functions through multiple pathways to reduce skin irritation and stabilize iontophoretic conditions. First, as a triprotic weak acid with pKa values of about 3.1, about 4.8, and about 6.4, citrate provides effective buffering capacity within the about 4.5 to about 6.5 pH window—precisely the range in which both NAD⁺ and BPC-157 exhibit optimal transport behavior with minimal charge antagonism. By resisting pH excursions at the electrode-skin interface, citrate limits the accumulation of protons (at the anode) or hydroxide ions (at the cathode) that would otherwise disrupt epidermal barrier integrity and provoke nociceptive responses. In unbuffered systems, these pH shifts are known to trigger localized acidosis or alkalosis, leading to erythema, stinging, or full-thickness burns, especially during extended wear periods typical of regenerative patch therapies.

Second, citrate ions act as mild chelators of divalent cations (e.g., calcium and magnesium) in the stratum corneum, which may otherwise precipitate under low pH and contribute to localized osmotic stress and irritation. Chelation moderates the electrochemical microenvironment, indirectly reducing inflammatory mediator release and improving barrier tolerability. Importantly, the inclusion of citrate at controlled concentrations also avoids excessive ionic competition, thereby preserving the electrokinetic driving force for NAD⁺ and BPC-157 transport.

Third, the citrate buffer system contributes to ionic strength modulation without overwhelming the current-carrying capacity of the patch. This is critical in dual-delivery systems, where electrokinetic efficiency must be maintained despite differing ion mobility profiles and molecular sizes. The citrate ions' intermediate mobility supports stable current distribution, avoiding abrupt impedance changes that could cause uneven delivery or skin resistance spikes—both of which compromise therapeutic consistency and user comfort.

Collectively, these features enable the disclosed formulation to address the inherent limitations of unbuffered iontophoresis systems and facilitate reproducible, irritation-minimized delivery of bioactive peptides and cofactors. The result is a stable, PH-balanced, electrochemically optimized delivery platform applicable to all compositions disclosed herein, including but not limited to NAD⁺, BPC-157, and their analogs or functional derivatives. Critically, the use of the anode as the delivery interface in this system reflects not only the charge compatibility of select subunits within each molecule, but also a deliberate exploitation of electroosmotic flow as a dominant transport mechanism—enabling effective delivery even for molecules with nominally unfavorable electrophoretic polarity. This integrated approach to electrokinetic design constitutes a significant advance over existing technologies by ensuring molecular compatibility, transport efficiency, and end-user tolerability within a single, unified patch system.

### Compositions

The present disclosure provides a composition comprising a combination of (a) NAD+, and (b) one or more (i) therapeutic peptides, (ii) peptide bioregulators, and/or (iii) compounds that are believed to facilitate cellular respiration and or regenerative cellular pathways, formulated for delivery via an iontophoretic system. In some embodiments, the compositions of the present disclosure are optimized for iontophoretic delivery. While iontophoresis offers a promising approach for transdermal delivery by utilizing an electric field to drive charged molecules through the skin, combining these specific agents in such a system presents significant technical challenges that render their integration neither straightforward nor obvious to one skilled in the art. These challenges stem from the need to balance multiple interdependent factors—pH, molecular charge states, skin tolerability, and electrode selection (anode or cathode)—each of which must be carefully optimized to ensure effective delivery and practical utility, as detailed below.

The compositions of the present disclosure include NAD+. NAD+ is believed to play an important role in the synthesis of adenosine triphosphate (ATP), an organic compound that provides energy for many processes in living cells, such as muscle contraction, nerve impulse propagation, condensate dissolution, and chemical synthesis, and as such NAD+ is a crucial nutrient for animal health.

In some embodiments, an amount of NAD+ in any of the compositions disclosed herein ranges from about 10% to about 30% by total weight of the composition. In other embodiments, the amount of NAD+ in the composition ranges from about 14% to about 25% by total weight of the composition. In other embodiments, the amount of NAD+ in the composition ranges from about 14% to about 20% by total weight of the composition. In other embodiments, the amount of NAD+ in the composition ranges from about 20% to about 25% by total weight of the composition. In yet other embodiments, the amount of NAD+ in the composition is about 10%, such as about 12%, such as about 14%, such as about 16%, such as about 18%, such as about 20%, such as about 22%, such as about 24%, such as about 26%, such as about 28%, such as about 30%, etc.

By way of example, a first composition may comprise about 250 mg of NAD⁺ (approximately 13.68% w/w), 2 mg of BPC-157 (approximately 0.11% w/w), 50 mg of sodium citrate (approximately 2.74% w/w), and 1.5 mL of water (approximately 82.08% w/w), yielding a total formulation mass of approximately 1.802 g. This composition prioritizes regenerative tissue signaling by combining the mitochondrial coenzyme NAD⁺ with the angiogenic and cytoprotective properties of BPC-157, while the inclusion of sodium citrate ensures pH buffering and ionic conductivity suitable for sustained iontophoretic delivery

By way of another example, a second composition may comprise about 250 mg of NAD⁺ (approximately 14.10% w/w), 10 mg of KPV tripeptide (approximately 0.56% w/w), 50 mg of sodium citrate (approximately 2.74% w/w), and 1.5 mL of water (approximately 84.55% w/w), yielding a total formulation mass of approximately 1.81 g. This formulation is optimized to support anti-inflammatory modulation in the context of energy-depleted or chronically inflamed tissues. The higher relative peptide content enhances localized immunomodulatory activity, while the citrate buffer again provides electrochemical stability and skin-compatible pH modulation necessary for effective iontophoretic transdermal transport.

The compositions of the present disclosure also include one or more (i) therapeutic peptides, (ii) peptide bioregulators, and/or (iii) compounds that are believed to facilitate cellular respiration and or regenerative cellular pathways. In some embodiments, the one or more (i) therapeutic peptides, (ii) peptide bioregulators, and/or (iii) compounds that are believed to facilitate cellular respiration and or regenerative cellular pathways are present in an amount ranging from between about 0.1 wt % to about 5.0 wt % by total weight of the composition. In some embodiments, the one or more (i) therapeutic peptides, (ii) peptide bioregulators, and/or (iii) compounds that are believed to facilitate cellular respiration and or regenerative cellular pathways are present in an amount ranging from between about 0.2 wt % to about 3.0 wt % by total weight of the composition. In other embodiments, the one or more (i) therapeutic peptides, (ii) peptide bioregulators, and/or (iii) compounds that are believed to facilitate cellular respiration and or regenerative cellular pathways are present in an amount ranging from between about 0.5 wt % to about 2.0 wt % by total weight of the composition. In yet other embodiments, the one or more (i) therapeutic peptides, (ii) peptide bioregulators, and/or (iii) compounds that are believed to facilitate cellular respiration and or regenerative cellular pathways are present in an amount ranging from between about 0.5 wt % to about 1.0 wt % by total weight of the composition. In further embodiments, the one or more (i) therapeutic peptides, (ii) peptide bioregulators, and/or (iii) compounds that are believed to facilitate cellular respiration and or regenerative cellular pathways are present in an amount ranging from between about 0.1 wt % to about 1.0 wt % by total weight of the composition.

In some embodiments, the therapeutic peptides are selected from:
ARA-290: Ac-CAEECRPIA-NH2 (denotes D-Ala)—Erythropoietin-derived peptide with tissue-protective and anti-inflammatory properties. (SEQ ID NO: 1)
BPC-157: GEPPPGKPADDAGLV—Body Protection Compound, promotes wound healing and tissue repair. (SEQ ID NO: 2)
B7-33: SLLGRMKGA—Immunomodulatory peptide derived from the CD80 protein. (SEQ ID NO: 3)
CJC-1295: YADAIFTNSYRKVLGQLSARKLLQDIMSR-OH—Growth hormone-releasing hormone (GHRH) analog with enhanced stability. (SEQ ID NO: 4)
DSIP: WAGGDASGE—Delta Sleep-Inducing Peptide, promotes sleep and has neuroprotective properties. (SEQ ID NO: 5)
FOXO4-DRI: D-Arg-Lys-Leu-His-D-Ala-NH2—Senolytic peptide that selectively eliminates senescent cells. (SEQ ID NO: 6)
GHK-Cu: GHK-Cu (Copper complex)—Tripeptide with wound healing and tissue regeneration properties.
GHRP-2: H-D-Ala-D-2-Nal-Ala-Trp-D-Phe-Lys-NH2—Growth Hormone-Releasing Peptide-2, stimulates growth hormone secretion ((D2Nal)=D-2-Naphthylalanine(.(SEQ ID NO: 7)
GHRP-6: His-DTrp-Ala-Trp-DPhe-Lys-NH2—Growth Hormone-Releasing Peptide-6, stimulates growth hormone secretion. (SEQ ID NO: 8)
GHRH: YADAIFTNSYRKVLGQLSARKLLQDIMSR-NH2—Growth Hormone-Releasing Hormone, stimulates growth hormone secretion. (SEQ ID NO: 9)
Glutathione: γ-Glu-Cys-Gly—Tripeptide with antioxidant and detoxification properties.
Humanin: MAPRGFSCLLLLTSEIDLPVKRRA—Mitochondrial-derived peptide with cytoprotective and neuroprotective properties. (SEQ ID NO: 10)
Ipamorelin: Aib-His-D-2-Nal-D-Phe-Lys-NH2—Growth hormone secretagogue with selective growth hormone release and minimal side effects.
Kisspeptin-10: YNWNSFGLRF-NH2—Peptide involved in the regulation of reproductive function and puberty onset. (SEQ ID NO: 11)
KPV: Lys-Pro-Val—Tripeptide derived from alpha-melanocyte-stimulating hormone (α-MSH) with anti-inflammatory properties.
LL-37: LLGDFFRKSKEKIGKEFKRIVQRIKDFLRNLVPRTES—Human cathelicidin antimicrobial peptide with immunomodulatory and wound healing properties. (SEQ ID NO: 12)
MOTS-c: MRWQEMGYIFYPRKLR—Mitochondrial-derived peptide with metabolic regulation and cytoprotective properties. (SEQ ID NO: 13)
Epithalon: AEDG—Tetrapeptide with geroprotective and neuroendocrine regulation properties.
Selank: TPLVTLFK-NH2—Synthetic anxiolytic and nootropic peptide derived from tuftsin. (SEQ ID NO: 14)
Semax: MEHFPGP—Synthetic heptapeptide with nootropic and neuroprotective properties. (SEQ ID NO: 15)
PE-22-28: VRSSSRT—Peptide derived from proenkephalin A with immunomodulatory properties. (SEQ ID NO: 16)
PNC-27: PPLSQETFSDLWKLLKKWKMRRNQFWVKVQRG—p53-derived peptide with antitumor properties. (SEQ ID NO: 17)
PNC-28: ETFSDLWKLLKKWKMRRNQFWVKVQRG—p53-derived peptide with antitumor properties. (SEQ ID NO: 18)
P21: KRRQTSMTDFYHSKRRLIFS—Cyclin-dependent kinase inhibitor peptide with potential antitumor properties. (SEQ ID NO: 19)
SS-31: D-Arg-Dmt-Lys-Phe-NH2—Mitochondria-targeted antioxidant peptide.
Thymosin Alpha-1: Ac-SDAAVDTSSEITTKDLKEKKEVVEEAEN-OH—Thymic peptide with immunomodulatory properties. (SEQ ID NO: 20)
Thymalin: Thymic peptides—Peptide complex with immunomodulatory and geroprotective properties.
Thyrotropin: Thyrotropin-releasing hormone (TRH)—Tripeptide that stimulates the release of thyroid-stimulating hormone (TSH).
VIP: HSDAVFTDNYTRLRKQMAVKKYLNSILN-NH2—Vasoactive Intestinal Peptide, neuromodulator, and vasodilator. (SEQ ID NO: 21)
TB4 (TB-500): Ac-SDKPDMAEIEKFDKSKLKKTETQEKNPLPSKETIEQEKQAGES-OH—Synthetic version of thymosin beta-4, promotes wound healing and tissue repair. (SEQ ID NO: 22)
Bronchogen (Ala-Glu-Asp-Leu): Lung peptide bioregulator—Tetrapeptide complex that supports lung function and regeneration.
Cardiogen (Ala-Glu-Asp-Arg): Heart peptide bioregulator—Tetrapeptide complex that supports heart function and regeneration.
Cartalax (Ala-Glu-Asp): Cartilage peptide bioregulator—Tripeptide complex that supports cartilage health and regeneration.
Chonluten (Glu-Asp-Arg): Lung peptide bioregulator—Tripeptide complex that supports lung function and regeneration.
Cortagen (Ala-Glu-Asp-Pro): Adrenal cortex peptide bioregulator—Tetrapeptide complex that supports adrenal cortex function and stress response.
Livagen (Lys-Glu-Asp-Ala): Liver peptide bioregulator—Tetrapeptide complex that supports liver function and regeneration.
Ovagen (Glu-Asp-Leu): Ovary peptide bioregulator—Tripeptide complex that supports ovarian function and reproductive health.
Pancragen (Lys-Glu-Asp-Trp): Pancreas peptide bioregulator—Tetrapeptide complex that supports pancreatic function and glucose metabolism.
Pinealon (Ala-Glu-Asp-Gly-NH2): Pineal peptide bioregulator—Amidated tetrapeptide that supports pineal gland function and circadian rhythm regulation.
Testagen (Lys-Glu-Asp-Gly): Testicle peptide bioregulator—Tetrapeptide complex that supports testicular function and male reproductive health.
Vesugen (Lys-Glu-Asp): Blood vessel peptide bioregulator—Tripeptide complex that supports vascular health and function.
Vesilute (Glu-Asp): Prostate peptide bioregulator—Dipeptide complex that supports prostate health and function.
Vilon (Lys-Thr-Lys-Lys-Glu-Ala-Ala-Lys-Lys-NH2): Geroprotective peptide bioregulator—Amidated nonapeptide with geroprotective and neuroendocrine regulation properties. (SEQ ID NO: 23)
Livagen: Liver peptide bioregulator—Peptide complex that supports liver function and regeneration.
Ovagen: Ovary peptide bioregulator—Peptide complex that supports ovarian function and reproductive health.
Pancragen: Pancreas peptide bioregulator—Peptide complex that supports pancreatic function and glucose metabolism.
Pinealon: AEDG-NH2—Tetrapeptide that supports pineal gland function and circadian rhythm regulation.
Testagen: Testicle peptide bioregulator—Peptide complex that supports testicular function and male reproductive health.
Vesugen: Blood vessel peptide bioregulator—Peptide complex that supports vascular health and function.
Vesilute: Prostate peptide bioregulator—Peptide complex that supports prostate health and function.

In some embodiments, the compounds that are believed to facilitate cellular respiration and or regenerative cellular pathways are selected from:
Methylene blue
Coenzyme Q10
Pyrroloquinoline quinone (PQQ)
Alpha-lipoic acid
Resveratrol
L-carnitine
Quercetin
Curcumin
Berberine
Oxaloacetic acid

In some embodiments, the compositions of the present disclosure include one or more additives. In some embodiments, the one or more additives are selected for pH adjustment and buffering to optimize iontophoretic delivery. Non-limiting examples of suitable additives are selected from the group consisting of sodium citrate, citric acid, sodium acetate, acetic acid, sodium bicarbonate, disodium hydrogen phosphate, tris base, and combinations thereof. Additives such as sodium citrate, citric acid, sodium acetate, and acetic acid are employed for buffering acidic solutions in the pH range of approximately 4 to 6, ensuring a positive charge state for compounds like NAD+ precursors or peptides during anode-driven iontophoresis, while maintaining skin compatibility. Additives such as sodium bicarbonate, disodium hydrogen phosphate, and tris base are utilized for buffering alkaline solutions in the pH range of approximately 7 to 8, facilitating neutral or negative charge states for cathode-driven delivery, with minimal risk of irritation. In some embodiments, the one or more additives are included within the composition in an amount ranging from about 0.5% to about 5% by total weight of the composition, providing a broad range to accommodate varying buffering needs and formulation stability. In other embodiments, the one or more additives are included within the composition in an amount ranging from about 1% to about 3% by total weight of the composition, reflecting a balanced concentration for effective pH control and iontophoretic compatibility, as exemplified by compositions containing approximately 1.57% to 3.37% sodium citrate. In yet other embodiments, the one or more additives are included within the composition in an amount ranging from about 3% to about 4.5% by total weight of the composition, tailored for higher buffering capacity in complex multi-agent systems while remaining safe for transdermal application.

| | Formulation | Formulation | Formulation | Formulation |
|---|---|---|---|---|
| | 1 | 2 | 3 | 4 |
| NAD+ | 13.68% w/w | 14.10% w/w | 13.71% w/w | NR |
| | | | | (24.36% w/w) |
| Additional | BPC-157 | KPV | TB500 | BPC-157 |
| active | (0.11% w/w) | (0.56% w/w) | (1.10% w/w) | (0.10% w/w) |
| agent 1 | | | | |
| Additional | | | BPC-157 | Insert name |
| active | | | (0.11% w/w) | and amount |
| agent 2 | | | | |
| Additive | Sodium | Sodium | Sodium | Sodium |
| 1 | citrate | citrate | citrate | citrate |
| | (2.74% w/w) | (2.74% w/w) | (2.74% w/w) | (1.23% w/w) |
| Additive | | | | Insert name |
| 2 | | | | and amount |
| pH | About | About | About | Insert pH |
| | 4.8-5.5 | 4.8-5.5 | 4.8-5.5 | range |

The present disclosure provides a method for enhancing and prolonging the regenerative effects of therapeutic peptides, peptide bioregulators, and/or compounds that facilitate cellular respiration and or regenerative cellular pathways by delivering them in combination with NAD+ via iontophoresis. The disclosure comprises the following key elements:

In some embodiments, the present disclosure comprises a synergistic combination of nicotinamide adenine dinucleotide (NAD+), one or more therapeutic peptides, peptide bioregulators, and compounds that facilitate cellular respiration and or regenerative cellular pathways, such as methylene blue. These components may be formulated together or provided separately for mixing prior to administration. Delivery methods, such as iontophoretic transdermal administration, are optimized to enhance bioavailability, ensuring each agent reaches target tissues in its active form. This design maximizes the efficacy of the combination by leveraging the complementary biological activities of each component to promote regenerative outcomes.

This disclosure harnesses the synergistic interplay of NAD+, therapeutic peptides, peptide bioregulators, and methylene blue to achieve regenerative effects greater than the sum of their individual contributions. The synergy stems from interconnected mechanisms that enhance mitochondrial function, cellular energy production, gene activation, and oxidative stress reduction. Below, I detail the roles of each component and their combined interactions, substantiated by citations from our conversation.

### i. Roles of Individual Components

NAD+: As a critical coenzyme, NAD+ drives ATP production via glycolysis, the TCA cycle, and oxidative phosphorylation. It also serves as a cofactor for sirtuins (e.g., SIRT1), which regulate mitochondrial biogenesis and cellular stress responses. Research shows that SIRT1 activation by NAD+ boosts energy efficiency through PGC-1α, enhancing mitochondrial density (Banks et al., 2008, Cell Metab 8, 333-341). Additionally, NAD+ supports DNA repair and cellular longevity, though its levels can be depleted by aging or stress (Bitterman et al., 2002, J Biol Chem 277, 45099-45107).
Therapeutic Peptides and Peptide Bioregulators: These molecules mimic endogenous signals to activate regenerative pathways. Peptide bioregulators, for instance, bind DNA to upregulate tissue-specific repair genes (Khavinson et al., 2003, Bull Exp Biol Med 135(1), 1-5). Therapeutic peptides like BPC-157 promote wound healing and angiogenesis by modulating growth factors (Gwyer et al., 2019, Front Bioeng Biotechnol 7, 187). Their effectiveness, however, relies on sufficient cellular energy and redox balance, which this combination supports.
Methylene Blue: This redox-active compound enhances mitochondrial respiration by acting as an electron carrier in the ETC, sustaining ATP production even under dysfunction (Atamna et al., 2008, FASEB J 22(3), 703-712). It also reduces oxidative stress by scavenging ROS, protecting cells during regeneration (Rojas et al., 2012, Prog Neurobiol 96(1), 32-45). These dual roles make it a key enabler of the combination's efficacy.

### ii. Synergistic Interactions

The synergy arises from complementary mechanisms, supported by the following evidence:
Boosted Mitochondrial Function and Energy Production: NAD+ enhances mitochondrial biogenesis via sirtuins (Banks et al., 2008), while methylene blue optimizes ETC efficiency (Atamna et al., 2008). Together, they ensure a steady ATP supply, critical for the energy demands of peptide-driven regeneration, such as protein synthesis and cell proliferation (Gwyer et al., 2019).
Amplified Gene Activation and Repair: Peptides and bioregulators trigger tissue repair by upregulating genes (Khavinson et al., 2003), but their success depends on cellular energy and mitochondrial health. NAD+ and methylene blue provide these conditions, amplifying the peptides' regenerative signals in energy-intensive tissues like muscle or brain.

Robust Oxidative Stress Defense: Regeneration is hindered by ROS, which damage cells and deplete NAD+ via PARP activation (Bürkle, 2005, FEBS J 272(18), 4576-4589). Methylene blue neutralizes ROS (Rojas et al., 2012), while NAD+-activated sirtuins upregulate antioxidants like MnSOD (Brunet et al., 2004, Science 303(5666), 2011-2015). This dual protection stabilizes the redox environment, sustaining repair processes.
Feedback Loops: NAD+-driven sirtuins improve mitochondrial function, reducing ROS and preserving NAD+ by limiting PARP activity (Bürkle, 2005). Methylene blue reinforces this by maintaining ETC efficiency (Atamna et al., 2008), creating a self-sustaining regenerative cycle.

### iii. Outcome of Synergy

This combination tackles regeneration's core challenges—energy, gene activation, and oxidative stress—more effectively than any single component. NAD+ and methylene blue enhance mitochondrial performance and redox balance, amplifying the regenerative potential of peptides and bioregulators. The result is faster tissue repair and greater cellular resilience, offering a novel approach for conditions like mitochondrial dysfunction or aging-related decline.
Iontophoresis Delivery: a. The combined formulation or individual components are delivered using an iontophoresis device, which can be either a traditional iontophoresis system or a self-contained iontophoresis patch. b. Traditional Iontophoresis Devices: i. Traditional iontophoresis devices consist of a power source, electrodes, a drug reservoir, a return reservoir, and a skin interface. ii. The power source, typically a battery or electrical outlet, provides the necessary current for ion transport. iii. The electrodes, usually a positive (anode) and a negative (cathode), establish an electrical circuit through the skin. iv. The drug reservoir contains the combined formulation or individual components to be delivered, while the return reservoir contains an electrolyte solution to complete the electrical circuit. v. The skin interface, often a conductive adhesive or gel, ensures proper contact between the electrodes and the skin. vi. During operation, the device is applied to the skin, with the drug reservoir in contact with the desired delivery site and the return reservoir at a distal location. vii. The power source generates a low-level electrical current, typically in the range of 0.1 to 1.0 mA/cm², which causes the charged molecules (NAD+, peptides, and other agents) to migrate through the skin and into the bloodstream. c. Self-Contained Iontophoresis Patches: i. Self-contained iontophoresis patches integrate all the necessary components of an iontophoresis device into a single, compact, and portable unit. ii. These patches consist of a power source, electrodes, a drug reservoir, a return reservoir, and a skin interface, all contained within a flexible and adhesive patch. iii. The power source is typically a thin, flexible battery that provides the necessary current for ion transport. iv. The electrodes, usually printed or embedded within the patch, establish an electrical circuit through the skin. v. The drug reservoir contains the combined formulation or individual components to be delivered, while the return reservoir contains an electrolyte solution to complete the electrical circuit. vi. The skin interface, often a conductive adhesive or gel, ensures proper contact between the patch and the skin. vii. To use the self-contained iontophoresis patch, the user simply applies the patch to the desired delivery site, ensuring that the drug reservoir is in direct contact with the skin. viii. The patch is activated, typically by pressing a button or removing a protective liner, which connects the power source to the electrodes and initiates the iontophoretic delivery process. ix. The self-contained patch delivers the combined formulation or individual components for the desired duration, typically about 3 to about 24 hours (such as about 4 to about 20 hours, such as about 6 to about 18 hours, such as about 6 to about 16 hours, etc.), providing a convenient and discreet method of administration. d. Both traditional iontophoresis devices and self-contained iontophoresis patches offer the advantage of controlled and sustained delivery of the therapeutic agents, allowing for a more consistent and prolonged effect compared to other routes of administration. e. The iontophoresis delivery system, whether traditional or self-contained, provides a means to overcome the limitations of short half-lives associated with NAD+, regenerative peptides, and other agents, ensuring a steady and effective concentration of these compounds in the bloodstream throughout the treatment period. f. The use of iontophoresis also allows for the localized delivery of the therapeutic agents to specific target sites, reducing the potential for systemic side effects and enhancing the efficacy of the treatment. g. The duration of iontophoretic delivery, typically 3 to 24 hours, can be adjusted based on the specific needs of the patient, the desired therapeutic effect, and the pharmacokinetic properties of the chosen compounds. h. The electrical current and other parameters of the iontophoresis device or patch can be optimized to ensure efficient and safe delivery of the combined formulation or individual components, while minimizing any potential discomfort or skin irritation.
Dosage and Formulation: a. The NAD+ dose ranges from between about 200 mg to about 1200 mg (such as about 300 mg to about 1100 mg, such as about 400 mg to about 1000 mg, such as about 500 mg to about 900 mg, such as about 600 mg to about 800 mg), based on an about 2 mL reservoir, with the dose scaled accordingly for other reservoir volumes. b. The therapeutic peptides, peptide bioregulators, and compounds that facilitate cellular respiration and or regenerative cellular pathways are dosed based on their specific chemical and pharmacokinetic properties, considering the desired therapeutic effect and duration of delivery. c. The dry powders or lyophilized components are mixed into a solution at the time of use to prevent degradation that could occur in pre-made solutions. d. A buffering agent, such as sodium citrate or disodium hydrogen phosphate, is included in the formulation to maintain a target pH that facilitates iontophoretic delivery while minimizing skin reactions due to the low pH of concentrated NAD+0 solutions.
Advantages and Applications: a. The synergistic combination of NAD+, regenerative peptides, peptide bioregulators, and/or compounds that facilitate cellular respiration and or regenerative cellular pathways, delivered via iontophoresis, offers enhanced and prolonged therapeutic effects compared to the administration of individual agents alone or sequentially. b. The sustained delivery system provided by iontophoresis overcomes the limitations of short half-lives associated with these agents, allowing for a more consistent and effective treatment. c. The optimization of cellular respiration by exogenous NAD+ and compounds like methylene blue throughout the delivery period creates a favorable environment for the regenerative processes mediated by the therapeutic peptides and bioregulators. d. The disclosure has potential applications in various fields of regenerative medicine, including wound healing, tissue repair, and the treatment of age-related degenerative conditions.

One of the key advantages of the present disclosure is its ability to overcome the limitations of traditional dose-response relationships associated with the short half-lives of NAD+, therapeutic peptides, peptide bioregulators, and/or compounds that facilitate cellular respiration and or regenerative cellular pathways. When delivered via conventional methods, such as oral administration or injection, these agents are rapidly metabolized and eliminated from the body, leading to a short duration of action and a limited cumulative biological effect. As a result, frequent dosing is required to maintain therapeutic levels, which can be inconvenient for patients and may lead to decreased compliance.

By utilizing iontophoresis for the transdermal delivery of these agents, the present disclosure enables an extended and relatively steady-state dose delivery, which prolongs the cumulative biological effects. The controlled and sustained release of NAD+ and regenerative agents from the iontophoretic device or patch allows for a more consistent and prolonged exposure of target tissues to these therapeutic compounds. This extended exposure facilitates a more robust and sustained activation of regenerative processes, such as cellular energy production, tissue repair, and angiogenesis.

Consequently, the iontophoretic delivery system described in this disclosure reduces the need for frequent dosing, as the steady-state delivery maintains therapeutic levels of the agents over an extended period. This not only enhances the overall biological effects but also improves patient compliance, as the burden of frequent administration is alleviated. Patients are more likely to adhere to the treatment regimen when the iontophoretic device or patch can be applied once daily or even less frequently, depending on the specific formulation and desired therapeutic outcomes.

Moreover, the transdermal delivery of NAD+ and regenerative agents via iontophoresis offers the advantage of avoiding potential risks associated with regular dosing via traditional methods that bypass first-pass metabolism. Oral administration of these compounds can lead to variable absorption and bioavailability due to individual differences in gastrointestinal function and first-pass hepatic metabolism. This can result in inconsistent therapeutic effects and potential side effects related to the metabolism of these agents in the liver.

In contrast, iontophoretic transdermal delivery allows for the direct absorption of the therapeutic agents into the systemic circulation, bypassing the gastrointestinal tract and first-pass metabolism. This direct absorption ensures a more predictable and consistent bioavailability of the compounds, reducing the variability in therapeutic responses among patients. Additionally, by avoiding first-pass metabolism, the iontophoretic delivery system minimizes the potential for liver-related side effects and drug-drug interactions that may occur with oral administration.

The composition's ability to provide extended and steady-state dose delivery, reduce dosing frequency, improve patient compliance, and avoid potential risks associated with traditional delivery methods that bypass first-pass metabolism further highlights the innovative and advantageous nature of the iontophoretic transdermal delivery system for NAD+ and regenerative agents. These benefits, combined with the synergistic effects of the carefully selected combination of compounds, position this disclosure as a significant advancement in the field of regenerative medicine, offering the potential for more effective, convenient, and safer therapeutic approaches for a wide range of regenerative applications.

In some embodiments, the present disclosure provides for a self-contained iontophoresis patch is designed to deliver a combination of about 500 to about 750 mg of NAD+, about 1 to about 5 mg of a therapeutic peptide (e.g., BPC-157 (where BPC-157 is the pentadecapeptide GEPPPGKPADDAGLV), about 1 to about 5 mg of a peptide bioregulator (e.g., Pinealon), and about 10 to about 50 mg of methylene blue over a duration of about 8 to about 24 hours. The patch is composed of a flexible, biocompatible polymeric substrate with an integrated power source, silver/silver chloride electrodes, and a drug reservoir containing the NAD+, peptide, bioregulator, and methylene blue formulation in a hydrogel matrix.

The inclusion of methylene blue or similar compounds that facilitate cellular respiration and or regenerative cellular pathways in the iontophoretic formulation further enhances the synergistic effects of NAD+ and the regenerative agents. Methylene blue acts as an electron donor and acceptor in the mitochondrial electron transport chain, improving mitochondrial function and energy production. This optimization of cellular respiration creates a favorable metabolic environment for the regenerative processes stimulated by the therapeutic peptides and bioregulators.

The patch is packaged in a sterile, ready-to-use form and can be easily applied to the skin at the desired site of delivery. Upon activation, the patch initiates the iontophoretic process, continuously delivering the therapeutic agents over the selected duration.

In the context of treating wounds or degenerative conditions, the iontophoresis patch can be applied directly to the affected area or in close proximity to optimize the local delivery of the regenerative agents. The sustained delivery of NAD+, peptides, bioregulators, and methylene blue over 8-24 hours provides prolonged support for tissue repair, angiogenesis, and the resolution of inflammation.

For systemic regenerative effects, the iontophoresis patch can be applied to a suitable site on the body, such as the upper arm or thigh, to facilitate the transdermal absorption of the therapeutic agents into the bloodstream. The continuous delivery of these agents over an extended period promotes their distribution to target tissues and organs, supporting regenerative processes throughout the body.

The self-contained iontophoresis patch provides a convenient and non-invasive method for the sustained delivery of NAD+, regenerative peptides, peptide bioregulators, and compounds that facilitate cellular respiration and or regenerative cellular pathways. This synergistic combination, delivered via iontophoresis, offers a comprehensive approach to regenerative medicine, addressing multiple aspects of tissue repair and regeneration, including energy production, cell signaling, inflammation, and oxidative stress.

In some embodiments, the present disclosure provides for a self-contained iontophoresis patch designed to deliver a combination of NAD+, a therapeutic peptide, a peptide bioregulator, and a therapeutic psychedelic compound for the treatment of traumatic brain injury (TBI) and post-traumatic stress disorder (PTSD). The patch is used in conjunction with a controlled therapeutic environment to facilitate repair, regeneration, and progressive therapy.

The iontophoresis patch is formulated to deliver about 500 to about 750 mg of NAD+, about 1 to about 5 mg of a therapeutic peptide (e.g., Selank or Semax), about 1 to about 5 mg of a peptide bioregulator (e.g., Cortexin or Pinealon), and a therapeutic psychedelic compound, such as ketamine, psilocybin, or LSD, at a dose ranging from a microdose to a full traditional psychedelic dose. The specific dose of the psychedelic compound is determined based on the individual patient's needs, tolerance, and response to treatment.

The patch is designed to deliver the combination of agents over a duration of about 8 to about 24 hours, providing a sustained and controlled release of the therapeutic compounds. The inclusion of NAD+ and the peptide components aims to optimize cellular respiration, support neuronal repair, and promote the regeneration of damaged neural tissue. The psychedelic compound, on the other hand, is included to facilitate neuroplasticity, enhance the therapeutic process, and address the psychological aspects of TBI and PTSD.

The treatment protocol involves the application of the iontophoresis patch in a controlled therapeutic environment, such as a specialized clinic or treatment center, under the supervision of trained medical professionals. The patient undergoes a comprehensive evaluation to determine the appropriate dosage of the psychedelic compound and to assess their suitability for the combined therapy.

Prior to the application of the patch, the patient participates in preparatory sessions with a therapist to establish a safe and supportive environment, discuss expectations, and develop a therapeutic framework. The iontophoresis patch is then applied, and the patient engages in a series of guided therapy sessions designed to maximize the potential benefits of the psychedelic experience while addressing the specific challenges associated with TBI and PTSD.

During the psychedelic experience, the patient may participate in various therapeutic activities, such as guided imagery, mindfulness practices, and trauma-focused interventions. The therapist provides support, guidance, and integration of the experience to help the patient process emotions, insights, and memories that may arise during the session.

Following the psychedelic session, the patient continues to wear the iontophoresis patch for the remainder of the designated time to maintain the sustained delivery of NAD+ and the peptide components. Integration sessions with the therapist are conducted to help the patient incorporate insights gained during the psychedelic experience into their daily life and to develop coping strategies for managing symptoms related to TBI and PTSD.

The combination of NAD+, therapeutic peptides, peptide bioregulators, and/or a psychedelic compound delivered via iontophoresis, coupled with a structured therapeutic environment, offers a multi-faceted approach to treating TBI and PTSD. The synergistic effects of these components may promote neuronal repair, facilitate neuroplasticity, and support the psychological healing process.

This embodiment highlights the potential of integrating the iontophoretic delivery of regenerative agents with psychedelic-assisted therapy to address the complex challenges associated with TBI and PTSD. By combining the physiological benefits of NAD+ and peptides with the psychological and neuroplastic effects of psychedelic compounds, this approach offers a comprehensive and innovative strategy for promoting healing and recovery in individuals affected by these conditions.

However, it is crucial to emphasize that the use of psychedelic compounds, even in a therapeutic context, should be conducted under strict medical supervision and in accordance with relevant legal and regulatory guidelines.

In some embodiments, the self-contained iontophoresis patch is specifically designed to deliver a combination of NAD+, BPC-157, and TB-500 (where TB-500, sequence SDKPDMAEIEKFDKSKLKKTETQEKNPLPSKETIEQEKQAGES is a 43 amino acid synthetic analogue of thymosin beta-4 (TB-4) for the treatment of myocardial infarction (MI) in critical care settings. The aim is to improve patient outcomes by promoting cardiac tissue repair, reducing inflammation, and supporting the recovery of heart function following an MI.

In some embodiments, the iontophoresis patch is formulated to deliver about 500 to about 750 mg of NAD+, about 1 to about 5 mg of BPC-157, and about 1 to about 5 mg of TB-500 over a duration of about 8 to about 24 hours. The combination of these agents is selected based on their potential synergistic effects in promoting cardiac tissue regeneration and mitigating the damaging consequences of MI.

NAD+ is included in the formulation to optimize cellular energy production, support mitochondrial function, and reduce oxidative stress in the cardiac tissue. BPC-157, a pentadecapeptide with well-established regenerative and cytoprotective properties, is incorporated to promote angiogenesis, reduce inflammation, and stimulate the repair of damaged cardiac tissue. TB-500, a synthetic version of the naturally occurring peptide thymosin beta-4, is included for its ability to promote wound healing, reduce inflammation, and improve cardiac function following injury.

The treatment protocol involves the application of the iontophoresis patch to the patient's chest area as soon as possible following the diagnosis of MI. In critical care settings, such as intensive care units or coronary care units, trained medical professionals apply the patch to ensure proper placement and initiate the iontophoretic delivery of the therapeutic agents.

The patch is designed to be compatible with standard monitoring equipment and can be used in conjunction with other necessary medical interventions, such as oxygen therapy, medications, and cardiovascular support devices. The sustained delivery of NAD+, BPC-157, and TB-500 over about 8 to about 24 hours allow for a prolonged therapeutic effect, providing support for the cardiac tissue during the critical phase of recovery following an MI.

Throughout the treatment period, patients are closely monitored for vital signs, cardiac function, and any potential adverse reactions. Blood tests, electrocardiograms, and imaging studies may be conducted to assess the patient's response to the therapy and to guide further treatment decisions.

The combination of NAD+, BPC-157, and TB-500 delivered via iontophoresis in critical care settings offers a novel approach to improving outcomes following MI. By providing a sustained and targeted delivery of these regenerative agents directly to the affected cardiac tissue, this embodiment aims to minimize the extent of cardiac damage, promote tissue repair, and support the recovery of heart function.

The potential benefits of this approach include:
Reduced infarct size and improved cardiac tissue salvage
Enhanced angiogenesis and revascularization of the affected area
Attenuated inflammation and oxidative stress
Improved cardiac function and hemodynamic stability.
Faster recovery and shortened hospital stay.
Reduced risk of complications and long-term disability

This embodiment highlights the potential of utilizing the iontophoretic delivery of NAD+, BPC-157, and TB-500 in critical care settings to improve outcomes for patients suffering from MI. By harnessing the regenerative and cytoprotective properties of these agents, this approach may provide a valuable adjunct to standard MI management, ultimately leading to better patient outcomes and quality of life.

In this embodiment, the self-contained iontophoresis patch is designed to deliver a combination of NAD+ or another compound intended to improve cellular respiration, and an anticholinergic drug for the treatment of various neurological, psychiatric, and gastrointestinal disorders, as well as for use as an anesthesia adjunct and for the treatment of motion sickness. The combination of these agents, delivered via iontophoresis, aims to create an improved therapeutic effect by enhancing cellular respiration while simultaneously modulating the activity of acetylcholine at muscarinic receptors.

The iontophoresis patch is formulated to deliver about 500 to about 750 mg of NAD+ or an alternative cellular respiration-enhancing compound, such as coenzyme Q10, pyrroloquinoline quinone (PQQ), or alpha-lipoic acid, and an anticholinergic drug at a dose tailored to the specific condition being treated. The anticholinergic drug may be selected from a diverse group, including but not limited to atropine, scopolamine, glycopyrrolate, hyoscyamine, or tiotropium, depending on the desired therapeutic effect and the patient's specific needs.

To ensure effective active transport of the anticholinergic drug via iontophoresis, the pH of the combination may be adjusted. The optimal pH range for iontophoretic drug delivery depends on the specific anticholinergic drug being used and its ionic charge. For example, atropine and scopolamine are basic drugs that are positively charged at physiological pH. In this case, the pH of the formulation may be adjusted to a slightly acidic range (e.g., pH 5.5-6.5) to promote the formation of the ionized form of the drug, thereby facilitating its active transport via iontophoresis. The pH adjustment can be achieved by incorporating suitable buffering agents, such as citric acid or phosphate buffers, into the formulation.

The patch is designed to deliver the combination of agents over a duration of about 8 to about 24 hours, providing a controlled and sustained release of the therapeutic compounds. This delivery method maximizes absorption while minimizing peak plasma levels, thereby reducing the risk of side effects associated with anticholinergic drugs, such as dry mouth, constipation, urinary retention, and cognitive impairment.

For the treatment of neurological and psychiatric disorders, such as Parkinson's disease, Alzheimer's disease, or schizophrenia, the iontophoresis patch is applied to a suitable site on the body, such as the upper arm or thigh, to facilitate the transdermal absorption of the therapeutic agents into the bloodstream. The sustained delivery of NAD+ or the alternative cellular respiration-enhancing compound supports neuronal function and energy metabolism, while the anticholinergic drug modulates the activity of acetylcholine in the central nervous system, potentially alleviating symptoms associated with these disorders.

In the context of gastrointestinal disorders, such as irritable bowel syndrome or chronic constipation, the iontophoresis patch is applied to the abdominal area to target the delivery of the therapeutic agents to the gastrointestinal tract. The combination of improved cellular respiration and anticholinergic effects may help to regulate gastrointestinal motility, reduce abdominal pain and discomfort, and alleviate constipation.

For the prevention and treatment of motion sickness, the iontophoresis patch is applied to a suitable site, such as behind the ear, to deliver the combination of NAD+ or the alternative cellular respiration-enhancing compound and an anticholinergic drug, such as scopolamine. The sustained release of these agents helps to minimize the symptoms of motion sickness, such as nausea, vomiting, and dizziness, by modulating the activity of acetylcholine in the vestibular system and improving cellular energy metabolism.

As an anesthesia adjunct, the iontophoresis patch is applied prior to surgical procedures to reduce the required doses of anesthetic agents and to minimize post-operative side effects. The combination of improved cellular respiration and anticholinergic effects may help to maintain hemodynamic stability, reduce post-operative nausea and vomiting, and promote a smoother recovery following anesthesia.

The self-contained iontophoresis patch offers a convenient and non-invasive method for the sustained delivery of NAD+ or alternative cellular respiration-enhancing compounds in combination with anticholinergic drugs. This innovative approach provides a targeted and controlled release of the therapeutic agents, potentially enhancing their efficacy while minimizing the risk of adverse effects associated with systemic administration. The pH adjustment of the formulation ensures optimal active transport of the anticholinergic drug via iontophoresis, further improving the delivery and therapeutic outcomes.

By leveraging the benefits of iontophoretic delivery, pH optimization, and the synergistic effects of cellular respiration enhancement and anticholinergic modulation, this embodiment offers a promising therapeutic strategy for a wide range of neurological, psychiatric, gastrointestinal, and motion sickness-related conditions, as well as for optimizing anesthesia management.

### Additional Embodiments

A method for comprising administering (i) one or more therapeutic peptides, peptide bioregulators, and/or compounds that facilitate cellular respiration and or regenerative cellular pathways, and (ii) NAD+, via iontophoresis.

The method of embodiment 1, wherein the NAD+, therapeutic peptides, peptide bioregulators, and compounds that facilitate cellular respiration and or regenerative cellular pathways are combined in a single formulation or as individual components to be mixed prior to administration.

The method of embodiment 1, wherein the iontophoresis delivery is achieved using a traditional iontophoresis device or a self-contained iontophoresis patch.

The method of embodiment 3, wherein the traditional iontophoresis device consists of a power source, electrodes, a drug reservoir, a return reservoir, and a skin interface.

The method of embodiment 3, wherein the self-contained iontophoresis patch integrates a power source, electrodes, a drug reservoir, a return reservoir, and a skin interface into a single, compact, and portable unit.

The method of embodiment 1, wherein the iontophoresis device or patch delivers the combined formulation or individual components for 3 to 24 hours, providing an extended therapeutic effect duration.

The method of embodiment 1, wherein the NAD+ dose range consists of 200 mg to 1200 mg, based on a 2 ml reservoir, with the dose scaled accordingly for other reservoir volumes.

The method of embodiment 1, wherein the therapeutic peptides, peptide bioregulators, and compounds that facilitate cellular respiration and or regenerative cellular pathways are dosed based on their specific chemical and pharmacokinetic properties.

The method of embodiment 1, wherein a buffering agent, such as sodium citrate or disodium hydrogen phosphate, is included in the formulation to maintain a target pH that facilitates iontophoretic delivery while minimizing skin reactions.

The method of embodiment 1, wherein the synergistic combination of NAD+, regenerative peptides, peptide bioregulators, and compounds that facilitate cellular respiration and or regenerative cellular pathways delivered via iontophoresis offers enhanced and prolonged therapeutic effects compared to the administration of individual agents alone or sequentially.

The method of embodiment 1, wherein the self-contained iontophoresis patch delivers a combination of about 500 to about 750 mg of NAD+, 1-5 mg of a therapeutic peptide, 1-5 mg of a peptide bioregulator, and about 10 50 mg of methylene blue over a duration of 8-24 hours for the treatment of wounds, degenerative conditions, or systemic regenerative support.

The method of embodiment 11, wherein the iontophoresis patch is applied directly to the affected area or in close proximity to optimize the local delivery of the regenerative agents.

The method of embodiment 11, wherein the iontophoresis patch is applied to a suitable site on the body, such as the upper arm or thigh, to facilitate the transdermal absorption of the therapeutic agents into the bloodstream for systemic regenerative effects.

1. A composition comprising (i) nicotinamide adenine dinucleotide (NAD+), and (ii) one or more therapeutic peptides and/or peptide bioregulators.

2. The composition of claim 1, wherein the composition comprises NAD+ in an amount ranging from between about 10% to about 30% by total weight of the composition.

3. The composition of claim 1, wherein the composition comprises NAD+ in an amount ranging from between about 14% to about 25% by total weight of the composition.

4. The composition of claim 1, wherein the one or more therapeutic peptides have any one of SEQ ID NOS: 1-23.

5. The composition of claim 1, wherein the one or more therapeutic peptides is Lys-Pro-Val (KPV).

6. The composition of claim 1, wherein the one or more therapeutic peptides has SEQ ID NO: 3.

7. The composition of claim 1, wherein the composition further comprises at least one of sodium citrate, citric acid, sodium acetate, or acetic acid.

8. The composition of claim 1, wherein the pH of the composition ranges from about 4.8 to about 5.5.

9. A patch comprising the composition of claim 1.

10. A method of reducing inflammation in a subject in need of treatment thereof, comprising administering to the subject the composition of claim 1.

11. A kit comprising (i) the composition of claim 1; and (ii) an iontophoretic delivery device.

12. The kit of claim 11, wherein the iontophoretic delivery device comprises a reservoir for storing the composition of claim 1.

13. New) The kit of claim 11, wherein the iontophoretic delivery device further comprises at least one electrode and an electrical energy source.

14. A composition comprising (i) nicotinamide adenine dinucleotide (NAD+), and (ii) one or more compounds selected from the group consisting of Methylene blue, Coenzyme Q10, Pyrroloquinoline quinone (PQQ), Alpha-lipoic acid, Resveratrol, L-carnitine, Quercetin, Curcumin, Berberine, and Oxaloacetic acid.

15. The composition of claim 14, wherein the composition comprises NAD+ in an amount ranging from between about 10% to about 30% by total weight of the composition.

16. The composition of claim 14, wherein the composition further comprises sodium citrate.

17. The composition of claim 14, wherein the pH of the composition ranges from about 4.8 to about 5.5.
